(19) United States
(12) Patent Application Publication
(10) Pub. No.: US 2021/0093594 A1
(43) Pub. Date: Apr. 1, 2021

(54) SKIN CONDITION IMPROVING AGENTS

(71) Applicant: SYMRISE AG, Holzminden (DE)

(72) Inventors: Gerhard SCHMAUS, Höxter-Bosseborn (DE); Sabine LANGE, Holzminden (DE); Paul SLAVASHEVICH, Bronx, NY (US)

(21) Appl. No.: 16/981,773

(22) PCT Filed: Mar. 25, 2019
(86) PCT No.: PCT/EP2019/057387

§ 371 Date: Sep. 17, 2020

Related U.S. Application Data
(60) Provisional application No. 62/647,158, filed on Mar. 23, 2018.

(30) Foreign Application Priority Data
Apr. 4, 2018 (EP) PCT/EP2018/058632

(51) Int. Cl. A61K 31/192; A61P 17/00; A61K 8/06; A61K 8/86; A61Q 19/00; A61K 9/107; A61K 47/10

(52) U.S. Cl. CPC A61K 31/192; A61P 17/00; A61K 8/062; A61K 2800/59; A61Q 19/007; A61K 9/107; A61K 47/10; A61K 8/86

(57) ABSTRACT

The present invention relates to a composition, preferably for use in a therapeutic method for improving or preventing one or more undesired skin conditions. Furthermore, the present invention relates to a cosmetic and/or dermatological preparation comprising such a composition.

The present invention relates to a composition comprising Avenanthramide(s) and/or polyalkylene glycol derivative(s) as well as oil bodies. One aspect of the present invention relates to such a composition for use in a therapeutic method for improving or preventing one or more undesired skin conditions. The present invention further relates to a cosmetic and/or dermatological preparation comprising such a composition.

Several skin diseases but also skin aging is associated with thinner, dry skin showing reinforced and undesired clinical symptoms like excoriation, lichenification and scaling.

Excoriation may typically be caused by excoriation disorder, with the onset of acne in adolescence or other skin conditions such as keratosis pilaris, psoriasis and eczema. Excoriation describes several skin lesions or abrasions caused by e.g. excessive grooming of the skin.

Lichenification describes a skin condition including epidermal thickening, typically characterized by visible and palpable thickening of the skin. Frequently, accentuated skin markings and/or an exaggeration of normal skin lines appear.

Scaling describes an abnormal shedding or accumulation of the upper layer of the skin (stratum corneum). Scaling is often associated with dry skin disorders.

The skin may tend to scaling e.g. because it is too dry. A physiological mechanism to prevent scaling by moisturizing the skin is the production of grease. However, with increasing age, use of e.g. soaps, shampoos, perfumes or disinfectants, contact with hot water and dry air conditions, e.g. caused by indoor heating, the skin may become dry, rough and start scaling. In general, substances causing de-fattening or degreasing and thus drying of the skin seem to promote the generation of undesired clinical symptoms such as e.g. excoriation, lichenification and dry skin (e.g. scaling).

WO 2016/207084A1 describes the use of polyethylene glycol esters and/or polyethylene glycol ethers in the treatment of itchy conditions of skin and scalp.

US 2010/150854A1 describes cosmetic and/or dermatological preparations containing polyethylene glycol esters and/or polyethylene glycol ethers for moisturizing the skin and to delay or prevent drying out of the skin.

DE 102 54 872 A1 relates to the use of Anthranilic acid amides as a cosmetic preparation for inhibiting the substance P induced release of Histamine. A reduction of Histamine release is reported to improve skin itching, pain and reddening of the skin.

However, further substances and cosmetic and/or dermatological preparations with increased effectiveness in improving or preventing undesired skin conditions are sought.

In the search for suitable agents it has to be considered that the substances used should be toxicologically acceptable, well tolerated by the skin and stable (in particular in conventional cosmetic and/or dermatological formulations) and they should preferably have the lowest possible intrinsic odour and the lowest possible intrinsic colour and be inexpensive to prepare.

The object of the present invention was therefore to develop a stable, cost-effective product with skin condition improving properties. Such skin conditions preferably comprise or consist of excoriation, lichenification and scaling associated with dry skin.

The object described above is solved by a composition according to the invention, comprising
a) one, two, three or more Avenanthramide(s) of formula (I)

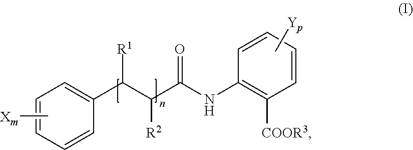

wherein
   m=0, 1, 2 or 3,
p=0, 1 or 2,
n=0, 1 or 2,
providing that if n is 1 or 2, the sum (p+m) is >0,
   wherein if n is 1 or 2, both R¹ and R² are H or together form a further chemical bond,
   wherein if m is 1, 2 or 3, each X is independent of the other(s) OH, O-Alkyl or O-Acyl,
   wherein if p is 1 or 2, each Y is independent of the other(s) OH, O-Alkyl or O-Acyl,
   providing that if (p+m)>0, X or Y is at least once selected from the group consisting of OH and O-Acyl,
   wherein R³=H, N or Alkyl,
and/or one, two, three or more polyalkylene glycol derivative(s) according to formula (II): R¹(OCH₂CHR²)ₙOR³,
in which
   R¹ stands for hydrogen or a linear or branched alkyl group with 1 to 4 carbon atoms,
   R² stands for hydrogen or methyl,
   n represents an integer of from 3 to 20
   R³ stands for a linear or branched alkyl or alkenyl group with 6 to 18 carbon atoms and 0, 1, 2 or 3 double bonds or a —COR⁴ acyl group, and
   R⁴ stands for hydrogen, a linear or branched alkyl or alkenyl group having 5 to 17 carbon atoms, or an alkali metal, an alkaline earth metal or NH₃
and optionally
b) oil bodies,
wherein
the ratio of the total amount of the Avenanthramide(s) and the total amount of the polyalkylene glycol derivative(s) in the composition, if present, is in the range of from 10:1 to 1:1000, preferably from 1:1 to 1:100, particularly preferably from 1:2 to 1:50, especially preferably from 1:5 to 1:25, based on weight.

The one, two, three or more Avenanthramide(s) of formula (I) in the composition according to the invention is/are or, respectively, comprise preferably one or two Avenanthramide(s) selected from the group consisting of Avenanthramides A, B, C, D and Dihydroavenanthramides A, B, C, D.

The one, two, three or more polyalkylene glycol derivative(s) of formula (II) in the composition according to the invention is/are or, respectively, comprise preferably one, two, three or more polyalkylene glycol derivative(s) according to formula (IIa)

H(OCH₂CH₂)ₙOR³ (IIa)

in which n represents an integer of from 7 to 12 and R³ stands for a linear or branched alkyl group with 8 to 15 carbon atoms,

and/or is/are preferably one, two, three or more polyalkylene glycol derivative according to formula (IIb)

H(OCH₂CH₂)ₙO—COR⁴ (IIb)

in which n represents an integer of from 3 to 7 and R⁴ stands for a linear or branched alkyl group with 7 to 11 carbon atoms.

The one, two, three or more polyalkylene glycol derivative(s) of formula (II) and/or (IIa) and/or (IIb) in the composition according to the invention is/are or, respectively, comprise preferably one, two, three or more polyethylene glycol (PEG) ether(s) of formula (IIa) and/or one, two, three or more polyethylene glycol (PEG) ester(s) of formula (IIb).

Polyethylene glycol ethers according to formula (II) and/or (IIa) that can be used in a composition are for example PEG-7 Octylether, PEG-7 Nonylether, PEG-7 Decylether, PEG-7 Undecylether, PEG-7 Dodecylether, PEG-7 Tridecylether, PEG-7 Tetradecylether, PEG-8 Octylether, PEG-8 Nonylether, PEG-8 Decylether, PEG-8 Undecylether, PEG-8 Dodecylether, PEG-8 Tridecylether, PEG-8 Tetradecylether, PEG-9 Octylether, PEG-9 Nonylether, PEG-9 Decylether, PEG-9 Undecylether, PEG-9 Dodecylether, PEG-9 Tridecylether, PEG-9 Tetradecylether, PEG-10 Octylether, PEG-10 Nonylether, PEG-10 Decylether, PEG-10 Undecylether, PEG-10 Dodecylether, PEG-10 Tridecylether, PEG-10 Tetradecylether, PEG-11 Octylether, PEG-11 Nonylether, PEG-11 Decylether, PEG-11 Undecylether, PEG-11 Dodecylether, PEG-11 Tridecylether, PEG-11 Tetradecylether, PEG-12 Octylether, PEG-12 Nonylether, PEG-12 Decylether, PEG-12 Undecylether, PEG-12 Dodecylether, PEG-12 Tridecylether, PEG-12 Tetradecylether.

Preferably, the PEG ether or one or two of the PEG ethers in the composition according to the invention is/are Polyoxyethylene (9) tridecyl ether (PEG-9 Tridecylether) and/or Polyoxyethylene lauryl ether (Laureth 9).

Polyethylene glycol esters that can be used in a composition according to formula (II) and/or (IIb) are for example PEG-3 Octanoate, PEG-3 Nonanoate, PEG-3 Isononanoate, PEG-3 Decanoate, PEG-3 Undecanoate, PEG-3 Dodecanoate, PEG-4 Octanoate, PEG-4 Nonanoate, PEG-4 Isononanoate, PEG-4 Decanoate, PEG-4 Undecanoate, PEG-4 Dodecanoate, PEG-5 Ethylhexanoate, PEG-5 3,5,5-Trimethylhexanoate, PEG-5 Octanoate, PEG-5 Nonanoate, PEG-5 Isononanoate, PEG-5 Decanoate, PEG-5 Undecanoate, PEG-5 Dodecanoate, PEG-6 Octanoate, PEG-6 Nonanoate, PEG-6 Isononanoate, PEG-6 Decanoate, PEG-6 Undecanoate, PEG-6 Dodecanoate, PEG-7 Octanoate, PEG-7 Nonanoate, PEG-7 Isononanoate, PEG-7 Decanoate, PEG-7 Undecanoate or PEG-7 Dodecanoate.

Preferably, the PEG ester or one or two of the PEG esters in the composition according to the invention is/are PEG-5 Ethylhexanoate and/or PEG-5 3,5,5-Trimethylhexanoate.

It is further preferred that the, one, two, three or more polyalkylene glycol derivative(s) is/are or, respectively comprise one, two or all polyalkylene glycol derivative(s) selected from the group consisting of PEG-5 Ethylhexanoate, Polyoxyethylene (9) tridecyl ether and PEG-5-3,5,5-Tri methyl hexanoate.

Oil bodies that can be used in a composition according to the invention are for example Guerbet alcohols based on fatty alcohols having 6 to 18, preferably 8 to 10, carbon atoms, esters of linear C6-C22-fatty acids with linear or branched C6-C22-fatty alcohols or esters of branched C6-C13-carboxylic acids with linear or branched C6-C22-fatty alcohols, such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl, palmitate, stearyl stearate, stearyl isostearate, stearyl oleate, stearyl behenate, stearyl eru-cate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C6-C22-fatty acids with branched alcohols, in particular 2-ethylhexanol, esters of C18-C38-alkylhy-droxy carboxylic acids with linear or branched C6-C22-fatty alcohols, in particular Dioctyl Malate, esters of linear and/or branched fatty acids with polyhydric alcohols (such as, for example, propylene glycol, dimerdiol or trimertriol) and/or Guerbet alcohols, triglycerides based on C6-C10-fatty acids, liquid mono-/di-/triglyceride mixtures based on C6-C18-fatty acids, esters of C6-C22-fatty alcohols and/or Guerbet alcohols with aromatic carboxylic acids, in particular benzoic acid, esters of C2-C12-dicarboxylic acids with linear or branched alcohols having 1 to 22 carbon atoms or polyols having 2 to 10 carbon atoms and 2 to 6 hydroxyl groups, vegetable oils, branched primary alcohols, substituted cyclohexanes, linear and branched C6-C22-fatty alcohol carbonates, such as, for example, Dicaprylyl Carbonate (Cetiol® CC), Guerbet carbonates, based on fatty alcohols having 6 to 18, preferably 8 to 10, carbon atoms, esters of benzoic acid with linear and/or branched C6-C22-alcohols (e.g. Finsolv® TN), linear or branched, symmetrical or asymmetrical dialkyl ethers having 6 to 22 carbon atoms per alkyl group, such as, for example, dicaprylyl ether (Cetiol® OE), ring-opening products of epoxidized fatty acid esters with polyols, silicone oils (cyclomethicones, silicone methicone grades, etc.) and/or aliphatic or naphthenic hydrocarbons, such as, for example, squalane, squalene or dialkylcyclohexanes.

In one embodiment of the present invention, component a) of the composition according to the invention consists of one, two, three or more Avenanthramide(s) of formula (I).

In this regard, component a) preferably consists of one distinct Avenanthramide. If not stated otherwise, said feature also applies for the paragraphs below.

In case several different Avenanthramides are present in the composition, the weight ratio of the Avenanthramide with the highest weight proportion and the, one or all other(s) Avenanthramide(s) is preferably from 10:1 to 1:10, preferably to more than 1:1, more preferably from 5:1 to 1:5, preferably to more than 1:1, and particularly preferably from 2:1 to 1:2, preferably to more than 1:1.

In another embodiment of the present invention, component a) of the composition according to the invention consists of one, two, three or more polyalkylene glycol derivative(s) of formula (II), wherein preferably the one, two, three or more polyalkylene glycol derivative(s) of formula (II) is/are or, respectively, comprise preferably one, two, three or more polyalkylene glycol derivative(s) according to formula (IIa) and/or formula (IIb).

In a preferred embodiment of the present invention, component a) of the composition according to the invention consists of one, two, three or more polyethylene glycol derivative(s) of formula (II), preferably of one, two, three or more polyethylene glycol ether(s) of formula (IIa) and one, two, three or more polyethylene glycol ester(s) of formula (IIb), particularly preferably of one polyethylene glycol ether of formula (IIa) and one polyethylene glycol ester of formula (IIb).

In case several different polyalkylene glycol derivatives are present in the composition, the weight ratio of the polyalkylene glycol derivative with the highest weight proportion and the, one or all other(s) polyalkylene glycol derivative(s) is preferably from 10:1 to 1:10, preferably to more than 1:1, more preferably from 5:1 to 1:5, preferably to more than 1:1, and particularly preferably from 2:1 to 1:2, preferably to more than 1:1.

In case several different polyethylene glycol derivatives, several different polyethylene glycol ethers and/or several different polyethylene glycol esters are present in the composition, said ratios apply accordingly and are preferred as well.

In a further embodiment of the present invention, component a) of the composition according to the invention consists of one, two, three or more Avenanthramide(s) of formula (I) and of one, two, three or more polyalkylene glycol derivative(s) of formula (II), preferably of formula (IIa) and/or formula (IIb).

It is particularly preferred that component a) consists of one Avenanthramide of formula (I) and of two polyalkylene glycol derivatives of formula (II).

Furthermore, it is especially preferred that component a) consists of one Avenanthramide of formula (I), one PEG ester and one PEG ether, preferably wherein
in the PEG ester the number of C-atoms of the polyethylene glycol part is 3 to 7 and/or the number of C-atoms of the ester part is 3 to 5
and/or
in the PEG ether the number of C-atoms of the polyethylene glycol part is 7 to 11 and/or the number of C-atoms of the ether part is 11 to 15.

Preferably, the, one, two or all polyalkylene glycol derivative(s) is/are selected from the group consisting of PEG-5 Ethylhexanoate, Polyoxyethylene (9) tridecyl ether and PEG-5-3,5,5-Trimethylhexanoate.

It is particularly preferred that component a) comprises or consists of Dihydroavenanthramide D, Polyoxyethylene (9) tridecyl ether and PEG-5 Ethylhexanoate. As described above, the Avenanthramide(s) can be one distinct or several different Avenathramide(s) and/or the polyalkylene glycol derivative(s) can be one distinct or several different polyethylene glycol ether(s) of formula (IIa) and/or one distinct or several different polyethylene glycol ester(s) of formula (IIb). In this regard, component a) preferably consists of one distinct Avenanthramide, one distinct polyethylene glycol ester and/or one distinct polyethylene glycol ether. If not stated otherwise, said feature also applies for the paragraphs below.

In another embodiment of the present invention, one or the, two, three or more polyalkylene glycol derivative(s) of the composition according to the present invention is/are or, respectively, comprise (a) polyethylene glycol ester(s), preferably wherein the number of C-atoms of the polyethylene glycol part is 3 to 7 and/or the number of C-atoms of the ester part is 3 to 5 and/or (a) polyethylene glycol ether(s), preferably wherein the number of C-atoms of the polyethylene glycol part is 7 to 11 and/or the number of C-atoms of the ether part is 11 to 15.

The term “polyethylene glycol part” of an ester or ether is meant to be understood as the part of the polyethylene glycol ester or ether which originates from polyethylene glycol.

The term “ester part” is meant to be understood as the part of the polyethylene glycol ester which originates from the acid, which formed an ester bond with polyethylene glycol resulting in the respective polyethylene glycol ester.

The term “ether part” is meant to be understood as the part of the polyethylene glycol ether which originates from the compound, which formed an ester bond with polyethylene glycol resulting in the respective polyethylene glycol ether. In a further embodiment of the present invention, one or the Avenanthramide, if present, is Dihydroavenanthramide D and/or the, one or more or all polyalkylene glycol derivative(s), if present, is/are or, respectively, comprise Polyoxyethylene (9) tridecyl ether and/or Polyoxyethylene lauryl ether.

In a preferred embodiment of the present invention, component a) comprises or consists of Dihydroavenanthramide D and Polyoxyethylene (9) tridecyl ether.

In a preferred embodiment of the present invention, component a) consists of Dihydroavenanthramide D and Polyoxyethylene (9) tridecyl ether.

In a preferred embodiment of the present invention, component a) comprises or consists of Dihydroavenanthramide D and Polyoxyethylene lauryl ether.

In a preferred embodiment of the present invention, component a) consists of Dihydroavenanthramide D and Polyoxyethylene lauryl ether.

In a further embodiment of the present invention, the one, two, three or more polyalkylene glycol derivative(s) is/are or, respectively, comprise one, two or all polyalkylene glycol derivative(s) selected from the group consisting of PEG-5 Ethylhexanoate, Polyoxyethylene (9) tridecyl ether and PEG-5-3,5,5-Trimethylhexanoate.

In one embodiment of the present invention, the total amount of component a) and/or b), if present, in the composition is sufficient to improve or prevent one or more undesired skin condition(s), wherein the undesired skin condition(s) is/are preferably one, two or more condition(s) selected from the group consisting of excoriation, lichenification and scaling associated with dry skin.

For the purposes of the present invention, “skin” is in particular the organ covering the outside of the human body and consisting of the epidermis, dermis and subcutis. Preferably, linings and/or coverings of internal organs, for example mucous membranes, periostea, vascular tissue and the retina are not “skin” for the purposes of the present invention.

The presence of polyalkylene glycol ester(s), particularly (a) polyethylene glycol ester(s) and (a) polyethylene glycol ether(s) in such a composition surprisingly showed a reduction in the clinical symptoms associated with dry skin such as scaling, excoriation and lichenification (see example 2).

Even more surprising, a combination of (an) Avenanthramide(s) and polyalkylene glycol ester(s), particularly (a) polyethylene glycol ester(s) and (a) polyethylene glycol ether(s) in such a composition shows reinforced reduction of the clinical symptoms associated with dry skin such as scaling, excoriation and lichenification (see example 3) although such effects hitherto have never been described for Avenanthramides.

In another embodiment of the present invention, the composition according to the invention can be used in a therapeutic method for improving or preventing one or more undesired skin condition(s), wherein the undesired skin condition(s) is/are preferably one, two or more condition(s) selected from the group consisting of excoriation, lichenification and scaling associated with dry skin.

A further aspect of the present invention relates to a cosmetic and/or dermatological preparation comprising a composition according to the invention, wherein such composition is present in a total amount sufficient to improve or prevent one or more undesired skin condition(s), wherein the undesired skin condition(s) is/are preferably one, two or more condition(s) selected from the group consisting of excoriation, lichenification and scaling associated with dry skin.

“Cosmetic” preparations for the purposes of the present invention are in particular preparations which are suitable for topical application to the skin of a human being, in particular to achieve a cosmetic effect. “Dermatological preparations” are preparations suitable for topical application to the skin of a human being to achieve a pharmaceutical effect, in particular to alleviate or cure a disease, in particular a skin disease.

It has also surprisingly been found that a composition according to the invention containing polyalkylene glycol derivative(s) has excellent solubilizing properties with regard to further, only moderately water-soluble, lipophilic substances or substance mixtures such as for example perfume oils, antidandruff agents, antiacne agents or substances regenerating the skin barrier. In particular, the very good solubility mediating properties of the composition according to the invention with regard to other active ingredients such as for example fatty oils, fatty acids, ceramides, pseudoceramides, sterols, phytosterols or hydrocarbons ideally suit them to be used in cosmetic and dermatological agents to improve or prevent one or more undesired skin conditions, in particular selected from the group consisting of excoriation, lichenification and scaling associated with dry skin.

In one embodiment of the present invention, the cosmetic and/or dermatological preparation is an oil-in-water (o/w) emulsion, preferably a two-phase system of an oil in water or a shampoo.

In another embodiment of the present invention, the total amount of polyalkylene glycol derivative(s), if present in the cosmetic and/or dermatological composition according to the invention, accounts for 0.1 to 5.0 wt.-%, preferably 0.5 to 2.5 wt.-%, of the preparation.

In a further embodiment of the present invention, the total amount of the Avenanthramide(s), if present in the cosmetic and/or dermatological composition according to the invention, accounts for 0.005 to 1.0 wt.-%, preferably 0.01 to 1.0 wt.-%, preferably 0.05 to 1.0 wt.-%, preferably 0.005 to 0.20 wt.-%, preferably 0.01 to 0.20 wt.-%, 0.02 to 0.20 wt.-%, of the preparation.

In another embodiment of the present invention, the cosmetic and/or dermatological preparation further comprises one or more moderately water-soluble active ingredients selected from the group consisting of odoriferous substances, perfume oils, aroma substances, aromas, fatty oils, fatty acids, waxes, ceramides, pseudoceramides, sterols, phytosterols, antiacne active ingredients, antidandruff active ingredients, antimicrobial active ingredients, preservatives, antiperspirants, antiirritants, pruritus-relieving active ingredients, cooling active ingredients, antioxidants, UV filters, antiaging active ingredients, skin-lightening and skin-tanning active ingredients, skin moisture regulators, osmolytes, insect repellents, enzyme inhibitors, odor absorbers, dyes.

In a further embodiment of the present invention, the cosmetic and/or dermatological preparation can be used in a therapeutic method for improving or preventing one or more undesired skin conditions, wherein the undesired skin condition(s) is/are preferably one, two or more conditions selected from the group consisting of excoriation, lichenification and scaling associated with dry skin.

The compounds as described herein are meant to be understood as the respective compounds, their stereoisomers and/or their respective salts.

Preferred embodiments and further aspects of the present invention emerge from the attached patent claims and the following examples, the examples not being intended to limit the scope of the present invention.

### EXAMPLES

### Example 1.1: Compositions According to the Invention

| | | Polyalkylene glycol | |
|---|---|---|---|
| | Avenanthramide | derivate(s) | Oil bodies |
| Substance | [wt.-%] | [wt.-%] | [wt.-%] |
| Composition 1 | 0.90 | 9.00 | 90.10 |
| Composition 2 | 0.24 | 4.80 | 94.96 |
| Composition 3 | 3.23 | 16.13 | 80.64 |
| Composition 4 | 1.64 | 16.39 | 81.97 |
| Composition 5 | 0.83 | 16.53 | 82.64 |
| Composition 6 | 2.04 | 16.33 | 81.63 |

### Example 1.2: Compositions According to the Invention

| | Dihydroavenant | Polyoxyethylene (9) | |
|---|---|---|---|
| | hramide D | tridecyl ether | Oil bodies |
| Substance | [wt.-%] | [wt.-%] | [wt.-%] |
| Composition 7 | 0.90 | 9.00 | 90.10 |
| Composition 8 | 0.24 | 4.80 | 94.96 |
| Composition 9 | 3.23 | 16.13 | 80.64 |

### Example 1.3: Compositions According to the Invention

| | Dihydroavenant | Polyoxyethylene | |
|---|---|---|---|
| | hramide D | lauryl ether | Oil bodies |
| Substance | [wt.-%] | [wt.-%] | [wt.-%] |
| Composition 10 | 0.90 | 9.00 | 90.10 |
| Composition 11 | 0.24 | 4.80 | 94.96 |
| Composition 12 | 3.23 | 16.13 | 80.64 |

### Example 1.4: Formulation Examples 1 to 14

Formulations (preparations) comprising compositions according to the invention having skin condition improving action:

1: Skin lightening day cream o/w
2: Every day shampoo
3: After sun balm
4: Deodorizing body spray
5: Sunscreen lotion (o/w, broadband protection)
6: w/o night cream
7: Anti dandruff shampoo
8: Anti-aging & barrier repair cream
9: Antiperspirant/deodorant roll-on

### 10: Refatting Liquid Soap

11: Refreshing hair conditioning spray

### 12: Shaving Cream o/w

13: Hair conditioner with UV-B/UV-A protection, rinse off
14: Hair conditioner, leave on

| | | % BY WEIGHT/FORMULATION EXAMPLE | | | | | | | | | | | | | |
|---|---|---|---|---|---|---|---|---|---|---|---|---|---|---|---|
| RAW MATERIAL | INCI | 1 | 2 | 3 | 4 | 5 | 6 | 7 | 8 | 9 | 10 | 11 | 12 | 13 | 14 |
| PEG-5 Isononanoate/ | | 0.6 | 0.6 | | 1.0 | | 1.2 | | | | 1.8 | | 0.1 | | 3.5 |
| PEG-9 Tridecylether | | 1.2 | 1.2 | 1.2 | 1.0 | 1.0 | 0.6 | 0.6 | | 0.1 | 0.2 | 0.2 | 1.9 | 1.9 | |
| PEG-5 Ethylhexanoate | | | 0.6 | 0.6 | | 1.0 | | 1.2 | | 1.9 | | 1.8 | | 0.1 | |
| Dihydro | | | 0.1 | | | | | | | | | 0.1 | | | |
| avenanthramide A | | | | | | | | | | | | | | | |
| Dihydro | | | 0.05 | | | | | | | | | | | | |
| avenanthramide B | | | | | | | | | | | | | | | |
| Dihydro | | | | | | | | | | 0.05 | | | | | |
| avenanthramide C | | | | | | | | | | | | | | | |
| Dihydro | | 0.1 | | 0.1 | 0.1 | 0.1 | 0.1 | 0.05 | 0.05 | 0.05 | | 0.2 | 0.2 | 0.02 | 0.1 |
| avenanthramide D | | | | | | | | | | | | | | | |
| Avenanthramide A | | | | | 0.1 | | | | | | | | | | |
| Avenanthramide B | | | | | | | | | 0.05 | | | | | | |
| Avenanthramide C | | | | | | | | | | 0.05 | | | | | |
| Avenanthramide D | | | | | | | | | | | 0.1 | | | | |
| Abil 350 | Dimethicone | 0.5 | | 1.0 | | | | | 0.5 | | | | | 0.1 | |
| Allantoin | Allantoin | | | 0.1 | | | | | 0.25 | | | | | | |
| Aloe Vera Gel | Water (Aqua). Aloe | | | 3.0 | | | 3.0 | 0.5 | | | | | | | |
| Concentrate 10/1 * | Barbadensis Leaf Juice | | | | | | | | | | | | | | |
| Alugel 34 TH | Aluminium Stearate | | | | | | 1.0 | | | | | | | | |
| Antil SPA 80 | Isostearamide MIPA. | | | | | | | | | | 1.0 | | | | |
| | GlcerylLaurate | | | | | | | | | | | | | | |
| Butylene Glycol | Butylene Glycol | | | 5.0 | | | | | | | | | | | |
| Carbopol Ultrez-10 | Carbomer | | | | | 0.2 | | | | | | | | | |
| Ceramide BIO* | Cetylhydroxyproline | | | | | | | 0.2 | 0.5 | | | | | | |
| | Palmitamide | | | | | | | | | | | | | | |
| Cetiol OE | Dicaprylyl Ether | | | 4.0 | | | | | | | | | | | |
| Cetiol SB 45 | Butyrospermum Parkii | | | 1.0 | | | | | | | | | | | |
| | (Shea Butter) | | | | | | | | | | | | | | |
| Citric Acid 10% sol. | Citric Acid | | 1.5 | | | | | 0.3 | | | 0.5 | | | | |
| Comperlan 100 | Cocamide MEA | | | | | | | 0.5 | | | | | | | |
| Crinipan AD | Climbazole | | | | | | | 0.5 | | | | | | | |
| Dehyquart A CA | Cetrimonium Chloride | | | | | | | | | | | 0.2 | | 0.2 | 0.5 |
| Dehyquart SP | Quaternium-52 | | | | | | | | | | | | | 0.5 | 4.0 |
| Dow Corning 246 | Cyclohexasilox-ane and | | | | | 2.0 | | | | | | | | | |
| Fluid | Cyclopentasilox-ane | | | | | | | | | | | | | | |
| Dow Corning 345 | Cyclomethicone | | | | 0.5 | | | | | | | | | | |
| Fluid | | | | | | | | | | | | | | | |
| D-Panthenol | Panthenol | | | 1.0 | | | | | | | | | 0.5 | | |
| Dracorin ® CE* | Glyceryl Stearate Citrate | 5.0 | | | | | | | 1.5 | | | | | 1.0 | 1.0 |
| Dracorin ® GOC* | Glyceryl Oleate Citrate. | | | | 2.0 | | | | | | | | | | |
| | Caprylic/Capric | | | | | | | | | | | | | | |
| | Triglyceride | | | | | | | | | | | | | | |
| Drago-Calm* | Water. Glycerin. Avena | 0.3 | | | | | | | | | | | | | 2.0 |
| | Sativa | | | | | | | | | | | | | | |
| | (Oat) Kernel Extract | | | | | | | | | | | | | | |
| Dragoderm ®* | Glycerin. Triticum | | | | | | | 2.0 | | | | | | | |
| | Vulgare (Wheat) | | | | | | | | | | | | | | |
| | Gluten. Water (Aqua) | | | | | | | | | | | | | | |
| Dragosan W/O P* | Sorbitan Isostearate. | | | | | | 6.0 | | | | | | | | |
| | Hydrogenated Castor Oil. | | | | | | | | | | | | | | |
| | Ceresin. Beeswax (Cera | | | | | | | | | | | | | | |
| | Alba) | | | | | | | | | | | | | | |
| Dragosantol ® 100* | Bisabolol | 0.3 | | | 0.1 | 0.3 | 0.2 | | | 0.1 | 0.1 | | 0.3 | | |
| Dragosine ®* | Carnosine | | | | | | | | 0.2 | | | | | | |
| Dragoxat ® 89 | Ethylhexyl Isononan-oate | | | | | | | | 2.0 | | | | | | |
| Edenor K12-18 | Coconut Palmkernel Oil | | | | | | | | | | | | 10.0 | | |
| | Fatty Acid | | | | | | | | | | | | | | |
| Edenor L2 SM | Stearic Acid. Palmitic | | | | | | | | | | | | 24.0 | | |
| | Acid | | | | | | | | | | | | | | |
| EDTA B | Tetrasodium EDTA | | | | | | | | | | | | 0.2 | | |
| EDETA BD | Disodium EDTA | | 0.1 | | | 0.1 | | | | | 0.1 | | | 0.1 | |
| Emulsiphos ® | Potassium Cetyl | | | | | 1.5 | | | 2.0 | | | | | | |
| | Phosphate. | | | | | | | | | | | | | | |
| | Hydrogenated Palm | | | | | | | | | | | | | | |
| | Glycerides | | | | | | | | | | | | | | |
| Ethanol 96% | Ethanol | | | | | | | | | 30.0 | 48.0 | | | | |
| Extrapone ® | Glycerin. Water (Aqua). | | | | | | | | 0.5 | | | | | | |
| Rosemary GW* | Rosmarinus officinalis | | | | | | | | | | | | | | |
| | (Rosemary) Leaf Extract | | | | | | | | | | | | | | |
| Extrapone ® | Propylene Glycol. | | | | | | 1.0 | | | | | | | | |
| Witch Hazel | Hamamelis Virginiana | | | | | | | | | | | | | | |
| Distillate colourless* | (Witch Hazel) Water. | | | | | | | | | | | | | | |
| | Water (Aqua). | | | | | | | | | | | | | | |
| | Hamamelis Virginiana | | | | | | | | | | | | | | |
| | (Witch Hazel) | | | | | | | | | | | | | | |
| | Extract | | | | | | | | | | | | | | |
| Farnesol* | Farnesol | | | | | | | | | 0.5 | | | | | |
| Fragrance* | Fragrance | 0.3 | 0.5 | 0.3 | 0.2 | 0.4 | 0.4 | 0.5 | 0.3 | 1.0 | 0.2 | 0.5 | 1.5 | 0.5 | 0.1 |
| Frescolat ® MGA* | Menthone Glycerol | 0.5 | | | | 0.3 | | | | | | | 0.5 | | |
| | Acetal | | | | | | | | | | | | | | |
| Frescolat ® ML cryst.* | Menthyl Lactate | | | 0.8 | | | | | | 0.2 | | | | | |
| Frescolat ® X-COOL* | Menthyl Ethylamido | | | | | | | | | | 0.5 | | | | |
| | Oxalate | | | | | | | | | | | | | | |
| Genapol LRO liquid | Sodium Laureth Sulfate | | | | | | | 37.0 | | | | | | | |
| Glycerin 99% | Glycerin | 3.0 | | 4.0 | | 4.7 | 2.0 | | 3.0 | | | | 2.0 | | |
| Glyceryl Stearate | Glyceryl Stearate | | | | | | | | 2.0 | | | | | | |
| Hydrolite ® -5 * | Pentylene Glycol | | | | 5.0 | | | | | | | 1.0 | | | |
| Hydroviton ® PLUS* | Water. Pentylene Glycol. | | | 1.0 | | | | | 1.0 | | | 1.0 | | | |
| | Glycerin. Fructose. | | | | | | | | | | | | | | |
| | Urea. Citric Acid. | | | | | | | | | | | | | | |
| | Sodium Hydroxide. | | | | | | | | | | | | | | |
| | Maltose. PCA. Sodium | | | | | | | | | | | | | | |
| | Chloride. Sodium | | | | | | | | | | | | | | |
| | Lactate. | | | | | | | | | | | | | | |
| | Trehalose. Allantoin. | | | | | | | | | | | | | | |
| | Sodium hyaluronate. | | | | | | | | | | | | | | |
| | Glucose | | | | | | | | | | | | | | |
| Isoadipate ® * | Diisopropyl Adipate | | | | | | | | | | 0.3 | 0.5 | | | |
| Isodragor ® * | Triisononanoin | | | | | | | | 3.0 | | | | | | |
| Isopropyl Palmitate | Isopropyl Palmitate | 4.0 | | | | | | | | | | | | | |
| Potassium Sorbate | Potassium Sorbate | | | | | | | | | | 0.2 | | | | |
| Karion F | Sorbitol | | | | | | 2.0 | | | | | | | | |
| Keltrol RD | Xanthan Gum | 0.2 | | | | | 0.2 | | | | | | | | |
| Lanette 16 | Cetyl Alcohol | 1.0 | | | | | | | | | | | | | |
| Lanette E | Sodium Cetearyl Sulfate | | | | | | | | | | | | | | |
| Lanette O | Cetearyl Alcohol | | | | | 1.0 | | | 2.0 | | | | | | |
| Lara Care A-200 | Galactoarabinan | | | 0.3 | | | | | | | | | | 0.5 | 1.5 |
| Magnesium Chloride | Magnesium Chloride | | | | | | 0.7 | | | | | | | | |
| Merquat 550 | Polyquaternium-7 | | | | | | | 0.5 | | | | | | | |
| NaOH 10% aqueous | Sodium Hydroxide | | | | | | | | 0.3 | | | | | | |
| sol. | | | | | | | | | | | | | | | |
| Natrosol 250 HHR | Hydroxyethyl-cellulose | | | | | | | | | 0.3 | | | | | |
| Neo Heliopan ® 357* | Butyl Methoxy- | | | | | 1.0 | | | | | | | | | |
| | dibenzoyl-methane | | | | | | | | | | | | | | |
| Neo Heliopan ® AP * | Disodium Phenyl | | | | | 10 | | | | | | | | | |
| (10% as sodium salt) | Dibenzimidazole | | | | | | | | | | | | | | |
| | Tetrasulfonate | | | | | | | | | | | | | | |
| Neo Heliopan ® AV* | Ethylhexyl Methoxy- | 5.0 | | | | 3.0 | | | | | | | | | |
| | cinnamate | | | | | | | | | | | | | | |
| Neo Heliopan ® | Isoamyl | | | | | 3.0 | | | | | | | | | |
| E1000* | p-Methoxycinnamate | | | | | | | | | | | | | | |
| Neo Heliopan ® HMS* | Homosalate | 2.0 | | | | | | | | | | | | | |
| Neo Heliopan ® | Phenylbenz-imidazole | | | | | 6.7 | | | | | | | | | |
| Hydro* | | | | | | | | | | | | | | | |
| (15% as sodium salt) | Sulfonic Acid | | | | | | | | | | | | | | |
| Neo Heliopan ® | 4-Methylbenzyl-idene | | | | | 1.5 | | | | | | | | | |
| MBC * | Camphor | | | | | | | | | | | | | | |
| Neo Heliopan ® OS* | Ethylhexyl Salicylate | | | | | 5.0 | | | | | | | | | |
| Neutral Oil | Caprylic/Capric | 6.0 | | | 4.0 | 2.0 | | | 10.0 | | | | 3.0 | | 1.0 |
| | Triglyceride | | | | | | | | | | | | | | |
| Paraffin Oil | Mineral Oil | | | | 4.0 | | | | | | | | | | |
| PCL Liquid 100* | Cetearyl Ethylhexoate | 3.0 | | | 7.0 | | 12.0 | | | | | | | | 0.3 |
| PCL Solid * | Stearyl Heptanoate. | | | | | | | | | | | | | 3.0 | |
| | Stearyl Caprylate | | | | | | | | | | | | | | |
| Pemulen TR-2 | Acrylates/C10-30 Alkyl | | | 0.3 | 0.2 | | | | | | | | | | |
| | Acrylate Crosspolymer | | | | | | | | | | | | | | |
| Plantacare PS10 | Sodium Laureth Sulfate. | | 17.0 | | | | | | | | | | | | |
| | Lauryl Glycoside | | | | | | | | | | | | | | |
| Polymer JR 400 | Polyquaternium-10 | | 0.2 | | | | | | | | | | | | 0.1 |
| Potassium Hydroxide | Potassium Hydroxide | | | | | | | | | | | | 11.0 | | |
| 50% aqueous sol. | | | | | | | | | | | | | | | |
| Propylene Glycol | Propylene Glycol | | | | | | | | | | | 0.8 | | | 0.8 |
| Sodium Ascorbyl | Sodium Ascorbyl | 2.0 | | 1.0 | | | | | | | | | | | |
| Phosphate | Phosphate | | | | | | | | | | | | | | |
| Sodium Benzoate | Sodium Benzoate | | | | | | | 0.5 | | | | | | | |
| Sodium Chloride | Sodium Chloride | | 0.4 | | | | | 1.0 | | | | | | | |
| Sodium Hydroxide | Sodium Hydroxide | | | 0.6 | 0.4 | | | | | | | | 5.0 | | |
| (10% sol.) | | | | | | | | | | | | | | | |
| Genapol LRO | Sodium Laureth Sulfate | | | | | | | | | | 20.0 | | | | |
| 28% AS | | | | | | | | | | | | | | | |
| Solubilizer 611674* | PEG-40 Hydrogenated | | | | | | | | | 2.0 | | | | | |
| | Castor Oil. Trideceth-9. | | | | | | | | | | | | | | |
| | Water (Aqua) | | | | | | | | | | | | | | |
| Sun Flower Oil | Helianthus Annuus | | | | | | 5.0 | | | | | | | | |
| | (Sunflower) Seed Oil | | | | | | | | | | | | | | |
| Sweet Almond Oil | Prunus dulcis | | | | | | 5.0 | | | | | | | | |
| SymCalmin ® | Pentylene Glycol. | | | 1.0 | | | | | 1.0 | | | | | | |
| | Butylene | | | | | | | | | | | | | | |
| | Glycol. Hydroxyphenyl | | | | | | | | | | | | | | |
| | Propamidobenzoic Acid | | | | | | | | | | | | | | |
| SymDeo ® B125* | 2-Methyl 5- | | | | 0.5 | | | | | 0.5 | | | | | |
| | Cyclohexylpentanol | | | | | | | | | | | | | | |
| SymDeo ® MPP* | Dimethyl Phenylbutanol | | | | 0.5 | | | | | | | | | | |
| Symdiol ® 68* | 1.2-Hexanediol. | | 1.0 | | | | | | | | 0.5 | | | | |
| | Caprylylglycol. | | | | | | | | | | | | | | |
| Symdiol ® 68T* | 1.2-Hexanediol. | 0.5 | | | | | | | | | | | | | |
| | Caprylylglycol. | | | | | | | | | | | | | | |
| | Tropolone | | | | | | | | | | | | | | |
| SymMatrix ®* | Maltodextrin. Rubus | | | | | 0.3 | 1.0 | | | | | | | | |
| | Fruticosus (Blackberry) | | | | | | | | | | | | | | |
| | Leaf Extract | | | | | | | | | | | | | | |
| SymMollient ® S * | Cetearyl Nonanoate | | | 1.0 | | | | | | | | | 1.5 | | |
| SymMollient ® WS * | PEG-5 Isononanoate | | 2.5 | | | | | | | | 0.5 | | | | |
| | (29%). Trideceth-9 | | | | | | | | | | | | | | |
| | (61%). Water (10%) | | | | | | | | | | | | | | |
| SymOcide ® PS * | Phenoxyethanol. | | | | | | | 1.0 | | | | | | | |
| | Decylene Glycol. | | | | | | | | | | | | | | |
| | 1.2 Hexanediol | | | | | | | | | | | | | | |
| Sympatens AL 090 | Laureth-9 | | | | 0.5 | | | | | | | | | 1.0 | |
| Sym Relief ® 100* | Bisabolol. Zingiber | | | | 0.1 | | | | | | | | 0.2 | | |
| | Officinale (Ginger) | | | | | | | | | | | | | | |
| | Root Extract | | | | | | | | | | | | | | |
| SymRepair ® 100* | Hexyldecanol. | | | | | | | | 2.0 | | | | | | |
| | Bisabolol. | | | | | | | | | | | | | | |
| | Cetylhydroxyproline | | | | | | | | | | | | | | |
| | Palmitamide. Stearic | | | | | | | | | | | | | | |
| | Acid. Brassica | | | | | | | | | | | | | | |
| | Campestris | | | | | | | | | | | | | | |
| | (Rapeseed) Sterols | | | | | | | | | | | | | | |
| SymSave ® H | Hydroxyyaceto-phenone | | 0.5 | | | | | | | | | | | | |
| SymSitive ® 1609 * | Pentylene Glycol. 4-t- | | | 1.5 | | | | | | | 0.5 | | | | |
| | Butylcyclohexanol | | | | | | | | | | | | | | |
| SymSol ® PF3 * | Water. Pentylene | | | | | | | | | | | | | | 1.5 |
| | Glycol. | | | | | | | | | | | | | | |
| | Sodium Lauryl | | | | | | | | | | | | | | |
| | Sulfoacetate. | | | | | | | | | | | | | | |
| | Sodium Oleoyl | | | | | | | | | | | | | | |
| | Sarcosinate. Sodium | | | | | | | | | | | | | | |
| | Chloride. Disodium | | | | | | | | | | | | | | |
| | Sulfoacetate. Sodium | | | | | | | | | | | | | | |
| | Oleate. Sodium Sulfate | | | | | | | | | | | | | | |
| SymVital ® | Zingiber Officinale | 0.1 | | | | | 0.1 | | | | | | | | |
| AgeRepair* | (Ginger) Root Extract | | | | | | | | | | | | | | |
| SymWhite ® 377* | Phenylethyl Resorcinol | 0.5 | | | | | | | | | | | | | |
| Tego Betain L7 | Cocamidopropyl | | 7.0 | | | | | 6.0 | | | 7.5 | 1.0 | | | |
| | Betaine | | | | | | | | | | | | | | |
| Tegosoft PC 31 | Polyglyceryl 3-Caprate | | | | | | | | 0.3 | | | | | | |
| Tegosoft TN | C12-15 Alkyl Benzoate | | | 5.0 | | 5.0 | | | | | | | | | |
| Tocopherol Acetate | Tocopheryl Acetate | | | 0.5 | | 0.5 | 3.0 | | 0.3 | | | | | | |
| Marlipal-13/99 | Trideceth-9 | | | 1.0 | | | | | | 2.5 | | | | | |
| Triethanolamine. 99% | Triethanolamine | | | | | 0.5 | | | | | | | | | |
| Water. demin. | Water (Aqua) | Ad 100 | | | | | | | | | | | | | |
| Zirkonal L 450 | Aluminium Zirconium | | | | | | | | | 37.0 | | | | | |
| | Pentachloro-hydrate | | | | | | | | | | | | | | |
| | (40% aqueous solution) | | | | | | | | | | | | | | |

### Example 2: In Vivo Application

Two preparations according to the invention were tested on 40 subjects (35-80 years) with visible dry skin. A double-blinded study was conducted for 4 weeks. The subjects successively used composition A and composition B, respectively, each of them for 2 weeks.

| | | Composition A | Composition B |
|---|---|---|---|
| Substance | INCL | [Wt.-%] | [Wt.-%] |
| NeoPcl ws | Trideceth-9, PEG-5 | 1.00 | 1.00 |
| | Ethylhexanoate | | |
| Water demin. | Water (Aqua) | 85.60 | 83.60 |
| SymCalmin | Butylene Glycol, Pentylene | — | 2.00 |
| | Glycol 1:1; 95%) | | |
| | Dihydroavenanthramide D | | |
| | (5%) | | |
| Euxyl K400 | Methyldibromoglutaronitrile, | 0.15 | 0.15 |
| | Phenoxyethanol | | |
| PCL liquid 100 | Cetearyl Octanoate | 3.00 | 3.00 |
| Lanette O | Cetearyl alcohol | 2.00 | 2.00 |
| Pemulen TR1 | Acrylates/C10-30 alkyl | 0.25 | 0.25 |
| | Actrylate Cross polymere | | |
| Paraffin oil 5° E | Mineral Oil | 3.00 | 3.00 |
| Eutanol G | Octyldodecanol | 4.00 | 4.00 |
| Abil 350 | Dimethicone | 0.50 | 0.50 |
| Sodium | Sodium hydroxide | 0.50 | 0.50 |
| hydroxide | | | |
| (10%) | | | |
| Sum | | 100.00 | 100.00 |
| pH | | 6.10 | 6.10 |

The parameters scaling, excoriation and lichenification were measured for the compositions A and B before the study (baseline), and after 2 weeks of application time, respectively. The parameters were assessed by applying a 10-point ordinal scale with 0=none up to 9=severe. The score reduction of preparation A or B, respectively, as listed below, was assessed as the score reduction between the measurements of baseline and after 2 weeks.

| | Scaling | | | Excoriation | | | Lichenification | | |
|---|---|---|---|---|---|---|---|---|---|
| | | Score | Score | | Score | Score | | Score | Score |
| | | reduc- | reduc- | | reduc- | reduc- | | reduc- | reduc- |
| | | tion vs. | tion vs. | | tion vs. | tion vs. | | tion vs. | tion vs. |
| | | Base- | Base- | | Base- | Base- | | Base- | Base- |
| | Score | line | line [%] | Score | line | line [%] | Score | line | line [%] |
| Base- | 6.35 | 0.00 | 100 | 5.73 | 0.00 | 100 | 5.53 | 0.00 | 100 |
| line | | | | | | | | | |
| A | 4.92 | 1.43 | 22.5 | 4.56 | 1.17 | 20.4 | 4.41 | 1.12 | 20.3 |
| B | 3.35 | 3.00 | 47.2 | 2.95 | 2.78 | 48.5 | 3.00 | 2.53 | 45.7 |

Preparation A, comprising Trideceth-9 and PEG-5 Ethylhexanoate reduces (mean values of 40 subjects) the clinical symptoms associated with dry skin such as scaling, excoriation and lichenification, respectively, by 22.5%, 20.4% and 20.3%, respectively

Preparation B, comprising Dihydroavenanthramide D, Trideceth-9 and PEG-5 Ethylhexanoate shows reinforced reduction (mean values of 40 subjects) of the clinical symptoms associated with dry skin such as scaling, excoriation and lichenification, respectively, by 47.2%, 48.5% and 45.7%, respectively.

### Example 3: In Vivo Application

Three shampoo preparations were tested on 20 subjects (16 females and 4 males; average age: 48.9 years) who suffered from scaling on the scalp.

| | Shampoo 1 | Shampoo 2 | Shampoo 3 |
|---|---|---|---|
| Substance | [Wt.-%] | [Wt.-%] | [Wt.-%] |
| Dihydroavenanthramide | 0.05 | 0.01 | — |
| D | | | |
| Trideceth-9 | — | 0.45 | 0.6 |
| PEG-5 Ethylhexanoate | — | 0.23 | 0.3 |

The study was carried out in a temperature and humidity-controlled room (24+/−2° C.; 50+10% R.U.). The subjects carried out a 7-day wash out period during which they used a standardized wash out shampoo without actives.

Subsequently, each volunteer tested shampoo 1, 2 or 3, according to a randomization chart. The products were filled in anonymous containers that did not provide any information about the treatment.

At the baseline, the volunteers were clinically examined for their degree of scaling (T₀). After the basal evaluations the shampoo (active or placebo) was given to the volunteers on the basis of the randomization chart. Each volunteer used the assigned shampoo three times a week.

A clinical evaluation about the scaling reduction of the skin was performed at the end of the treatment (T₇).

| | Shampoo 1 | Shampoo 2 | Shampoo 3 |
|---|---|---|---|
| Reduction of scaling | [Wt.-%] | [Wt.-%] | [Wt.-%] |
| Score T₇/T₀ | 1 | 0.778 | 1 |

In subjects treated with shampoo 1 or 3, no reduction of scaling could be observed. Patients treated with shampoo 2, however, showed a reduction of their scaling of 22.2%. This was surprising, particularly, as shampoo 2 contains even less of the single substances than shampoo 1 or, respectively, shampoo 3. This finding was furthermore unexpected, as the sum of Dihydroavenanthramide D, Trideceth-9 and PEG-5 Ethylhexanoate in shampoo 2 is even less than the sum of Trideceth-9 and PEG-5 Ethyl hexanoate in shampoo 3.

1-15. (canceled)

16. A composition comprising:
(a) one or more avenanthramides of Formula (I) and/or one or more polyalkylene glycol derivatives of Formula (II): 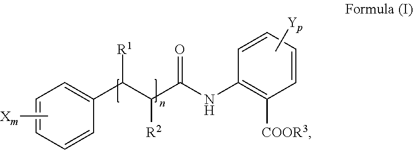
wherein in Formula (I),
m=0, 1, 2, or 3,
p=0, 1, or 2,
n=0, 1, or 2,
provided that if n is 1 or 2, the sum (p+m) is >0,
wherein, if n is 1 or 2, both R¹ and R² are H or together form a further chemical bond,
if m is 1, 2 or 3, each X is independently OH, O-alkyl, or O-acyl, and
if p is 1 or 2, each Y is independently OH, O-alkyl or O-acyl,
provided that if (p+m)>0, at least one of X and Y is OH or O-acyl,
wherein R³=H, N or alkyl;
R¹(OCH₂CHR²)ₙOR³ Formula (II),
wherein in Formula (II),
R¹ is hydrogen or a linear or branched alkyl group with 1 to 4 carbon atoms,
R² is hydrogen or methyl,
n represents an integer of from 3 to 20,
R³ is a linear or branched alkyl or alkenyl group with 6 to 18 carbon atoms and 0, 1, 2 or 3 double bonds or a —COR⁴ acyl group, and
R⁴ is hydrogen, a linear or branched alkyl or alkenyl group having 5 to 17 carbon atoms, or an alkali metal, an alkaline earth metal or NH₃; and
(b) optionally, oil bodies;
wherein the weight ratio of the total amount of the one or more avenanthramides to the total amount of the one or more polyalkylene glycol derivatives in the composition, if present, is from 10:1 to 1:1000.

17. The composition according to claim 16, wherein component (a) consists of the one or more avenanthramides of Formula (I) and the one or more polyalkylene glycol derivatives of Formula (II).

18. The composition according to claim 17, wherein the one or more polyalkylene glycol derivatives of Formula (II) are polyethylene glycol esters.

19. The composition according to claim 17, wherein component (a) consists of one avenanthramide of Formula (I) and two polyalkylene glycol derivatives of Formula (II).

20. The composition according to claim 17, wherein component (a) consists of one avenanthramide of Formula (I) and two polyalkylene glycol derivatives of Formula (II), wherein one polyalkylene glycol derivative is a PEG ester and one polyalkylene glycol derivative is a PEG ether.

21. The composition according to claim 17, wherein the one or more avenanthramides of Formula (I) includes dihydroavenanthramide D and the one or more polyalkylene glycol derivatives of Formula (II) includes polyoxyethylene (9) tridecyl ether and/or polyoxyethylene lauryl ether.

22. The composition according to claim 17, wherein the one or more polyalkylene glycol derivatives of Formula (II) are selected from PEG-5 ethylhexanoate, polyoxyethylene (9) tridecyl ether, and PEG-5-3,5,5-trimethylhexanoate, and mixtures thereof.

23. The composition according to claim 17, wherein the total amount of component (a) in the composition is sufficient to improve or prevent one or more undesired skin condition(s) selected from excoriation, lichenification, and scaling associated with dry skin.

24. The composition according to claim 16 that is a cosmetic or dermatological composition that improves or prevents one or more undesired skin conditions(s) selected from excoriation, lichenification, and scaling associated with dry skin.

25. The composition according to claim 24 in the form of an oil-in-water (o/w) emulsion.

26. The composition according to claim 24 comprising 0.1 to 5.0 wt. % of the one or more polyalkylene glycol derivatives of Formula (II), based on the total weight of the composition.

27. The composition according to claim 24 comprising 0.005 to 1.0 wt. % of the one or more avenanthramides of Formula (I), based on the total weight of the composition.

28. The composition according to claim 24, further comprising one or more active ingredients selected from odoriferous substances, perfume oils, aroma substances, aromas, fatty oils, fatty acids, waxes, ceramides, pseudoceramides, sterols, phytosterols, antiacne active ingredients, antidandruff active ingredients, antimicrobial active ingredients, preservatives, antiperspirants, antiirritants, pruritus-relieving active ingredients, cooling active ingredients, antioxidants, UV filters, antiaging active ingredients, skin-lightening and skin-tanning active ingredients, skin moisture regulators, osmolytes, insect repellents, enzyme inhibitors, odor absorbers, dyes, and mixtures thereof.

29. A composition according to claim 16 comprising:
(a) dihydroavenanthramide D and two polyalkylene glycol derivatives of Formula (II),
wherein the weight ratio of the total amount of the dihydroavenanthramide D to the total amount of the one or more polyalkylene glycol derivatives of Formula (II) in the composition is from 1:2 to 1:50;
(b) an oily phase; and
(c) water;
wherein the composition is in the form of an oil-in-water (o/w) emulsion.

30. The composition according to claim 29 comprising 0.005 to 1.0 wt. % of the dihydroavenanthramide D, based on the total weight of the composition.

31. The composition according to claim 29 comprising 0.1 to 5.0 wt. % of the one or more polyalkylene glycol derivatives of Formula (II), based on the total weight of the composition.

32. A method for improving or preventing one or more undesired skin conditions comprising applying to the skin the composition according to claim 16.

33. The method according to claim 32, wherein the one or more undesired skin conditions are selected from excoriation, lichenification, and scaling associated with dry skin.

34. A method for improving or preventing one or more undesired skin conditions comprising applying to the skin the composition according to claim 24.

35. The method according to claim 34, wherein the one or more undesired skin conditions are selected from excoriation, lichenification, and scaling associated with dry skin.
